(19) United States
(12) Patent Application Publication
(10) Pub. No.: US 2009/0076445 A1
(43) Pub. Date: Mar. 19, 2009

(54) Sterile catheter pullback mechanism assemblies

(75) Inventor: Simon M. Furnish, New York, NY (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 12/219,169

(22) Filed: Jul. 17, 2008

Related U.S. Application Data
(60) Provisional application No. 60/929,919, filed on Jul. 18, 2007.

(51) Int. Cl. A61M 25/092

(52) U.S. Cl. 604/95.01

(57) ABSTRACT

One aspect of the invention provides catheter pullback apparatuses in which a reusable drive component is enclosed in a sterile disposable barrier enclosure, such as a polymeric sheath/bag. In one embodiment, the reusable drive component includes a pullback carriage that is magnetically coupled, through the sterile barrier, to a slideable catheter shuttle adapted to retain a catheter hub of a catheter. In another embodiment, a sterile barrier-enclosed drive component is inserted into a housing, in which process a piercing tip provided on the catheter shuttle pierces the enclosure and mechanically mates the slideable catheter shuttle and pullback carriage. In this manner, non-sterility of the operating environment is minimized.

### CROSS-REFERENCE TO RELATED APPLICATIONS

This application claims the benefit of U.S. provisional patent application Ser. No. 60/929,919 filed Jul. 18, 2007, which is incorporated by reference herein in its entirety.

### FIELD OF THE INVENTION

The invention relates generally to the field of endoluminal catheters.

### BACKGROUND OF INVENTION

Catheter pullback mechanisms are used to provide controlled longitudinal displacement of catheters, such as intravascular catheters, during diagnostic and/or treatment procedures. Various catheter pullback mechanisms have been described in the art.

U.S. Pat. No. 6,814,727 discloses automatic/manual longitudinal position translator and rotary drive system for catheters, and is incorporated herein by reference in its entirety.

U.S. Pat. No. 6,506,144 discloses a pullback mechanism for an in-situ treatment device such as a catheter, and is incorporated by reference herein in its entirety.

In view of the above, what is needed are improved catheter pullback devices that better maintain sterility within the vicinity of use while permitting re-use of core components.

### SUMMARY OF INVENTION

The invention overcomes the drawbacks and disadvantages of the prior art by providing catheter pullback (PB) mechanism assemblies in which a reusable drive component is enclosed by a sterile barrier enclosure, such as a polymeric sheath/bag.

One embodiment of the invention provides a catheter pullback apparatus, including:

a reusable elongate drive unit comprising a pullback carriage;

a sterile barrier enclosure sized and configured to surround the reusable elongate drive unit;

a base unit sized and configured to receive the reusable elongate drive unit surrounded by the disposable sterile barrier enclosure; and

a catheter shuttle slideably engaged with the base unit and magnetically coupleable to the pullback carriage of the reusable elongate drive unit.

Another embodiment of the invention provides a catheter pullback apparatus, including:

a reusable elongate drive unit comprising a pullback carriage;

a sterile barrier enclosure sized and configured to surround the reusable elongate drive unit;

a base unit sized and configured to receive the reusable elongate drive unit surrounded by the disposable sterile barrier enclosure; and

a catheter shuttle comprising a barrier piercing member and slideably engaged with the base unit, said shuttle mechanically coupleable to the pullback carriage of the reusable elongate drive unit by the barrier piercing member.

The sterile barrier enclosures may be single-use, disposable items, for example, polymeric disposable polymeric sleeves or bags. Preferably, the base unit is sterilizable and reusable, so that both the base unit and drive unit may be re-used.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

### Magnetically Coupled Embodiment Drawings

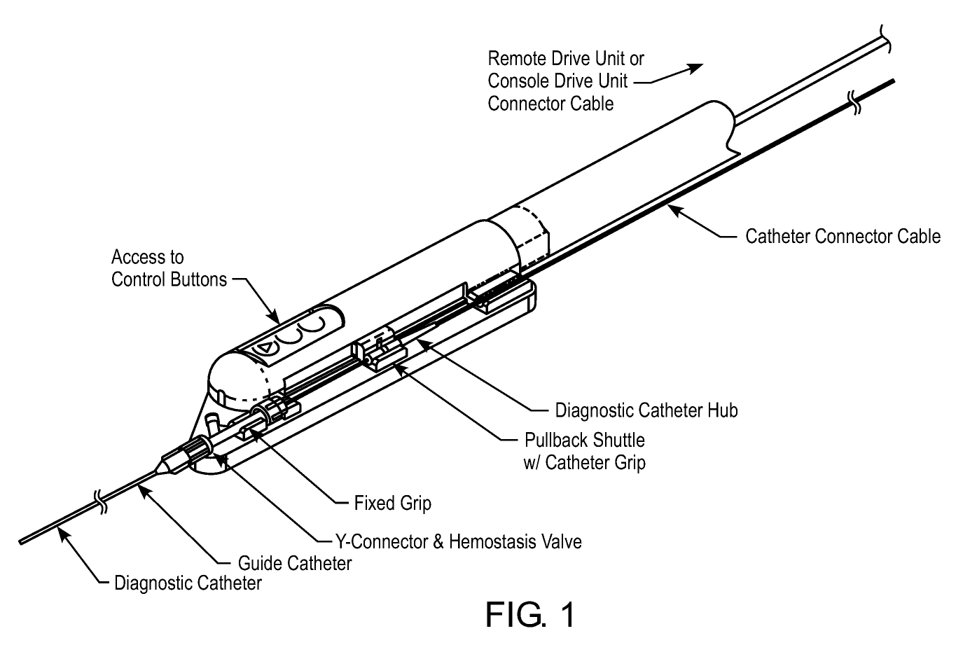

FIG. 1: Sterile PB Device Assembly. Isometric view of full assembly with catheter (and accessories), sterile base assembly, sterile sleeve and reusable motor drive assembly

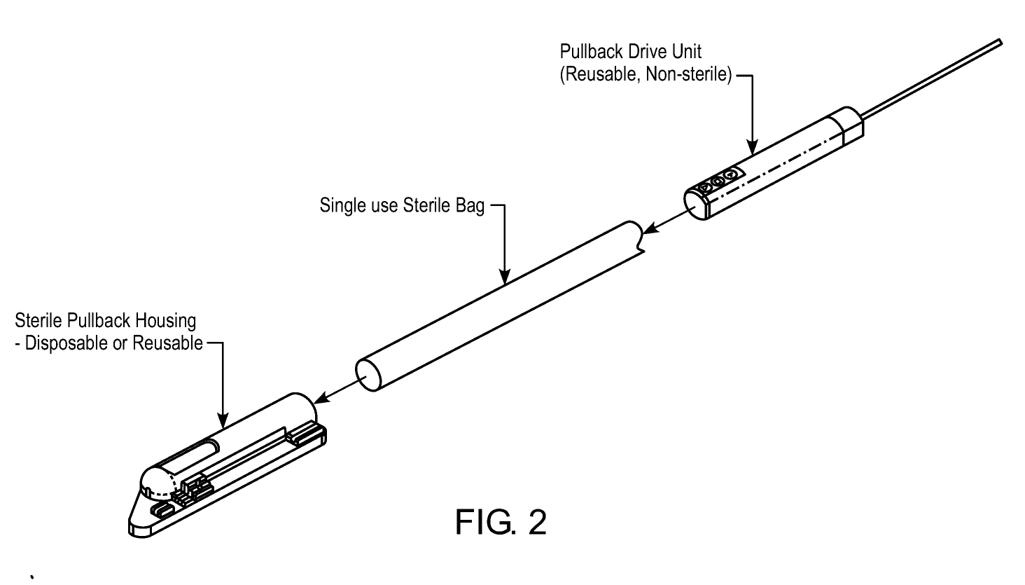

FIG. 2: Sterile PB Device Assembly. Isometric exploded view showing Sterile pullback housing, single-use sterile bag (sleeve) and reusable Motor/Pullback Drive Unit.

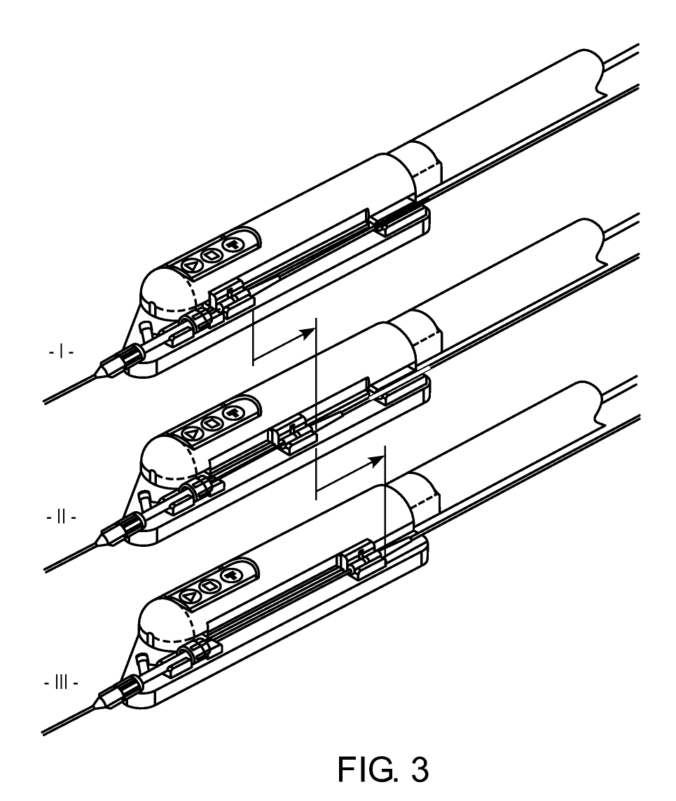

FIG. 3: Sterile PB Device Assembly. Isometric view showing motor drive assembly & catheter in three sequential positions: I. Start; II. Mid-pullback; and III. Full Pullback.

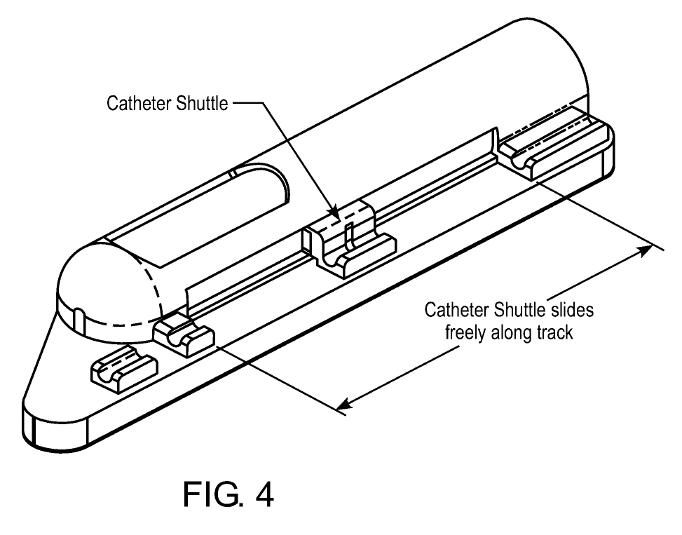

FIG. 4: Isometric view of sterile pullback housing assembly with catheter shuttle and track.

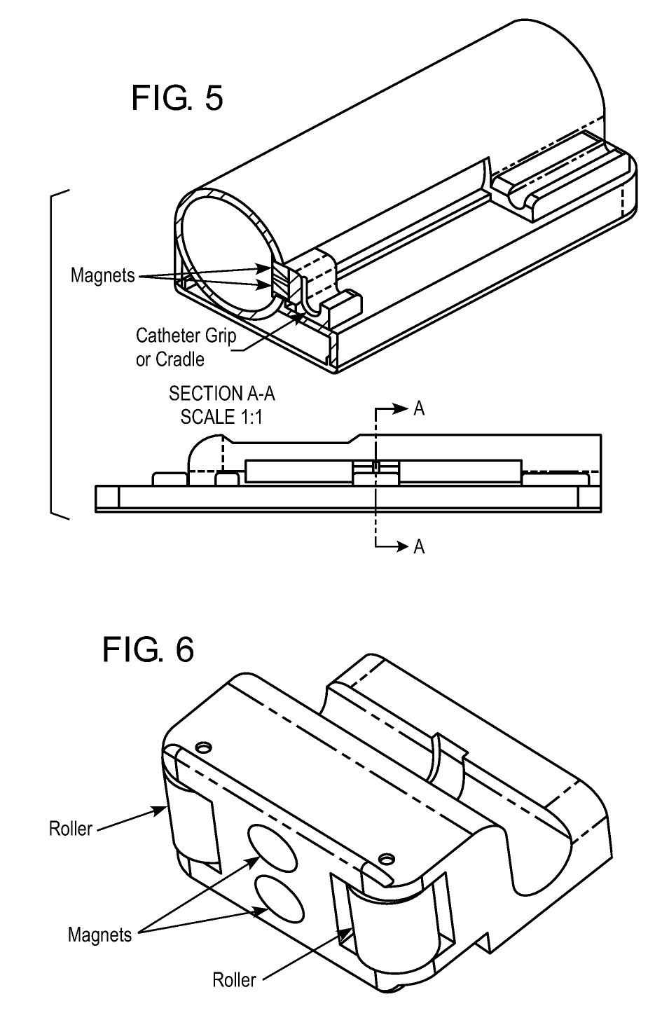

FIG. 5: Sterile PB Device Assembly. Sectional isometric view through Catheter Shuttle showing magnetic coupling details.

FIG. 6: Isometric view of pullback shuttle embodiment with rollers and magnets.

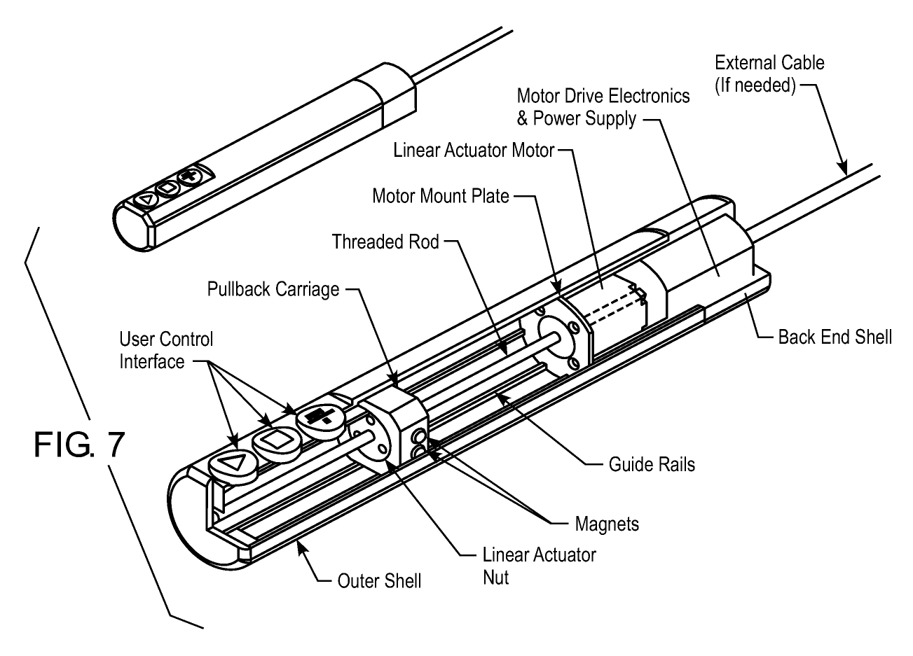

FIG. 7: Isometric view of reusable magnetic drive motor pullback assembly with cut-away.

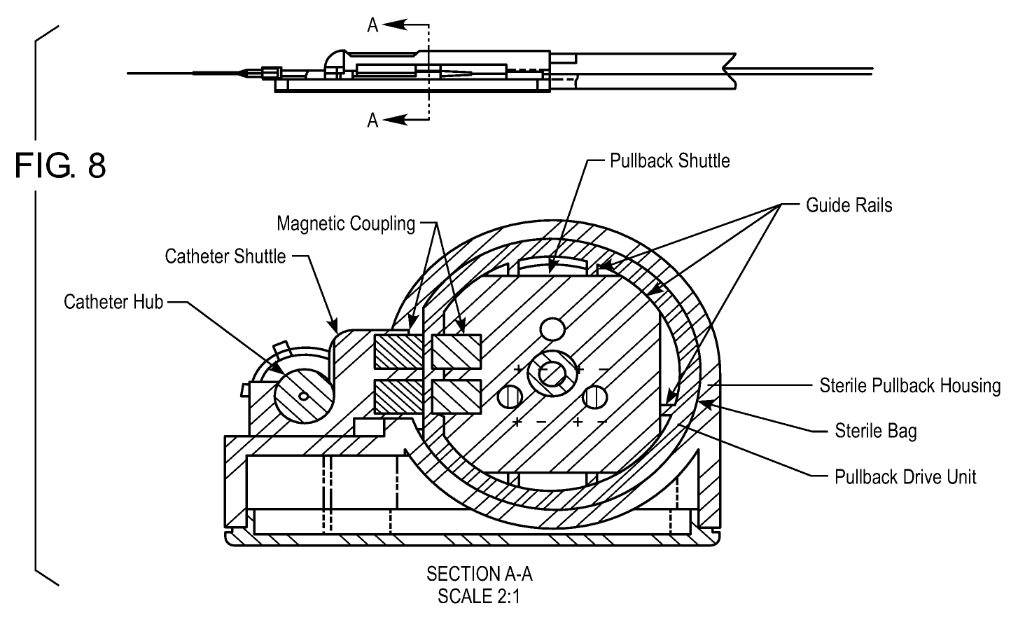

FIG. 8: Section view through fully assembled pullback unit with catheter showing magnetic coupling between pullback shuttle and catheter shuttle.

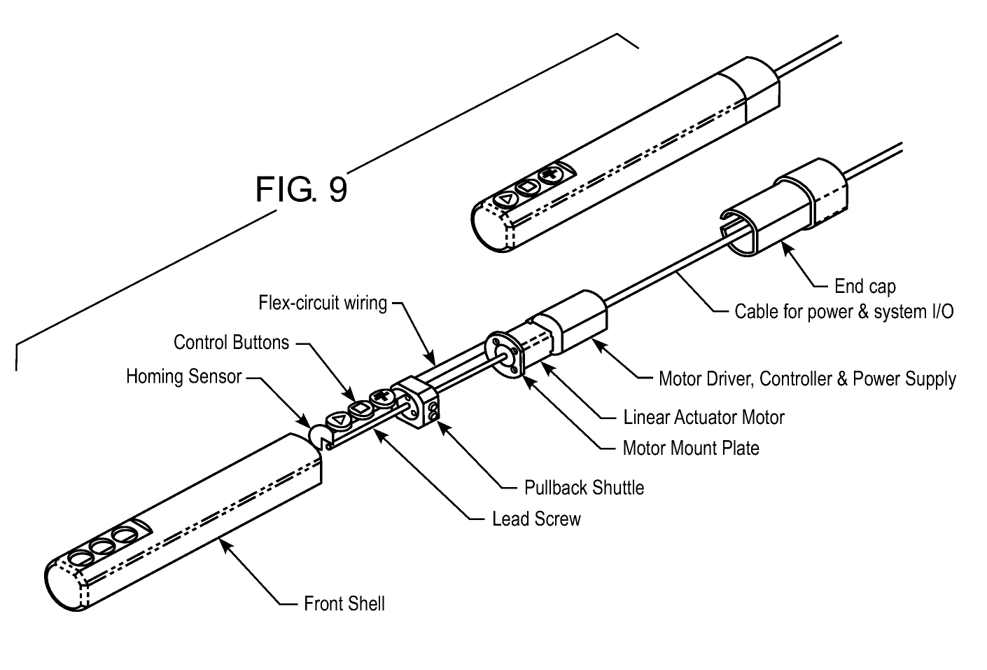

FIG. 9: Isometric exploded view of magnetically coupled reusable pullback motor assembly.

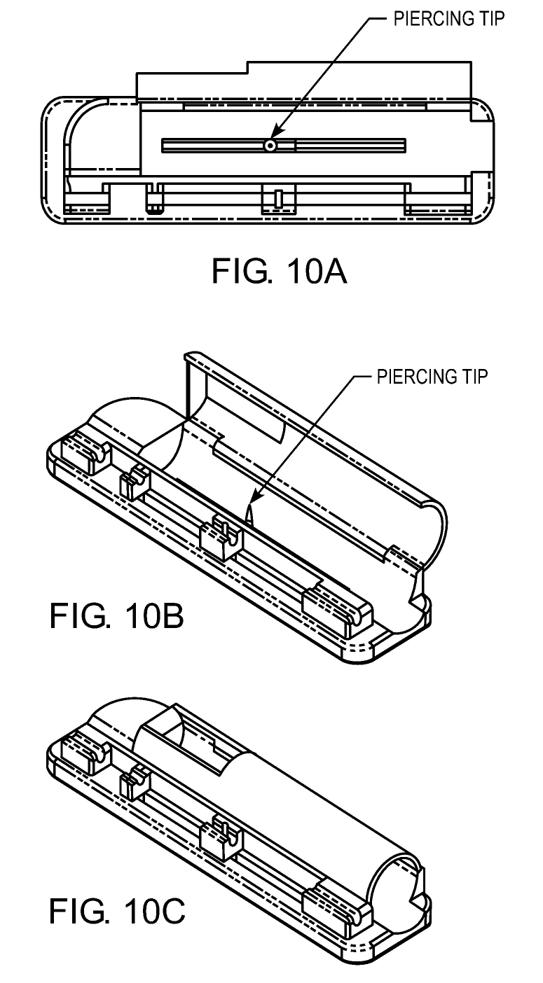

FIG. 10: Sterile PB Device Assembly. Top view shows piercing tip inside pullback slot. Isometric views shown with lid open and closed positions.

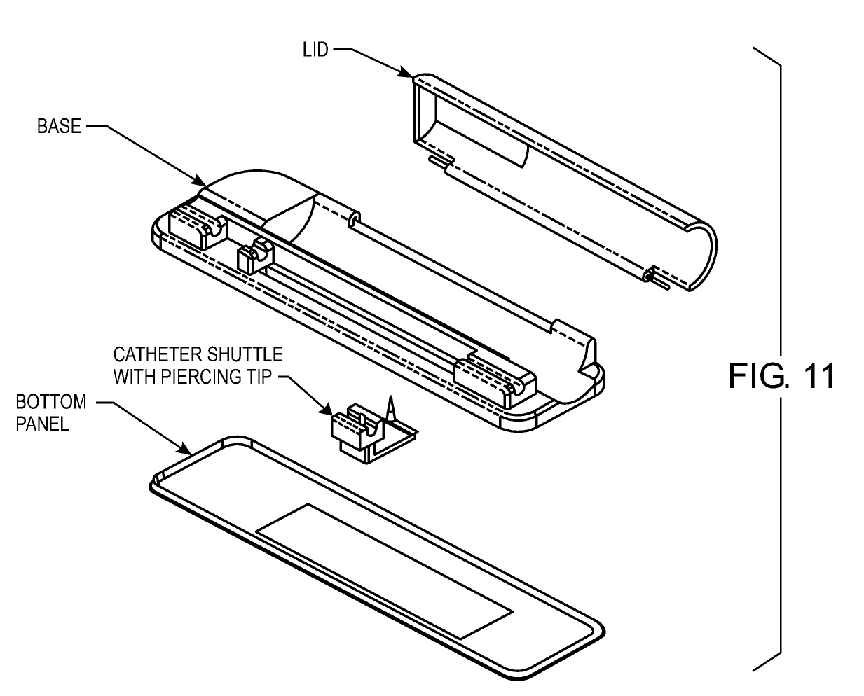

FIG. 11: Sterile PB Device Assembly. Isometric exploded view showing lid, base, catheter shuttle and bottom panel components. Shuttle slides back and fort along slot & guides provided on base and bottom panel.

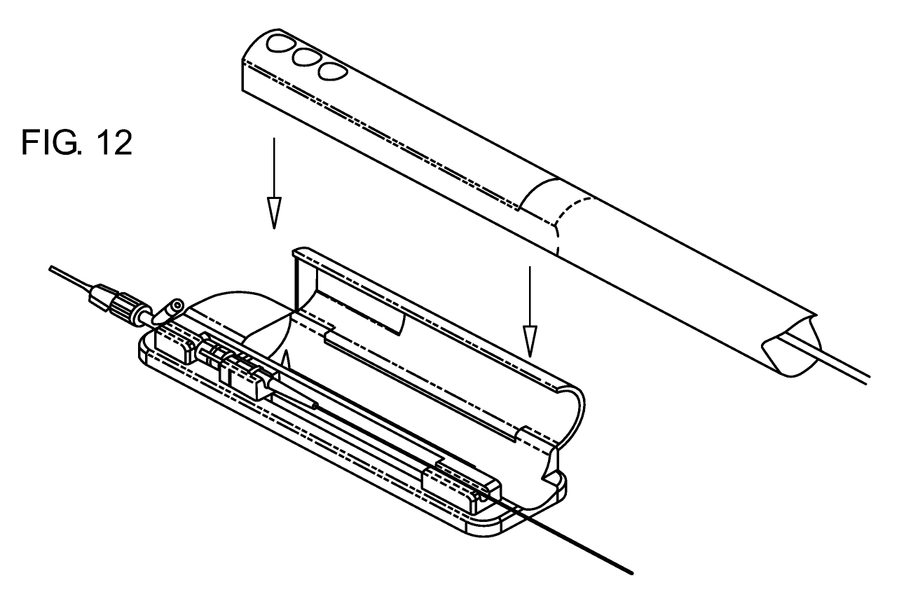

FIG. 12: Sterile PB Device Assembly. Isometric view showing motor drive assembly within sterile sleeve inserted from above. Sharp tip will puncture sleeve when motor drive unit is seated in the sterile base assembly.

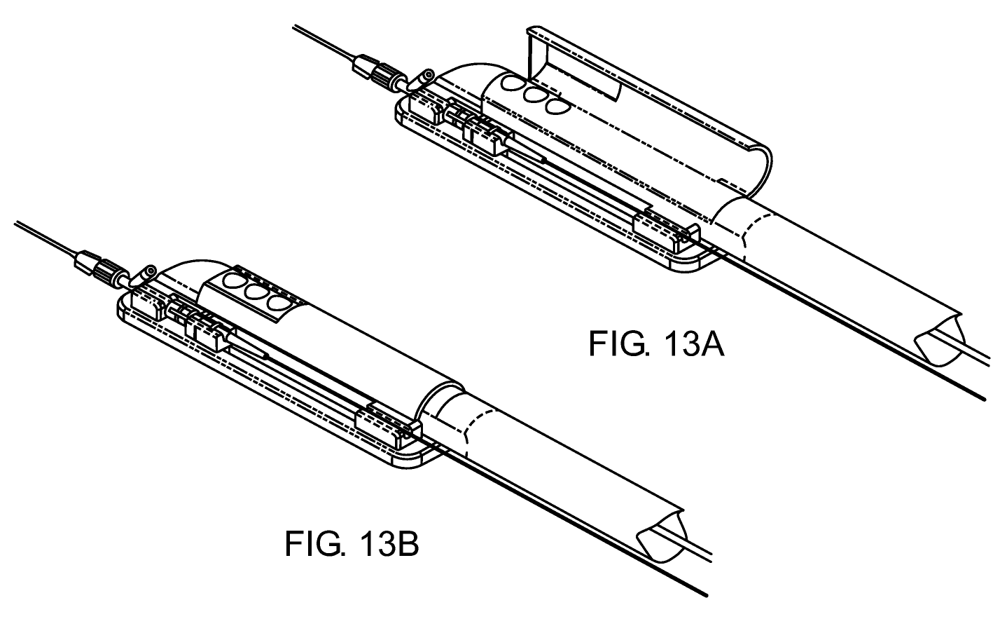

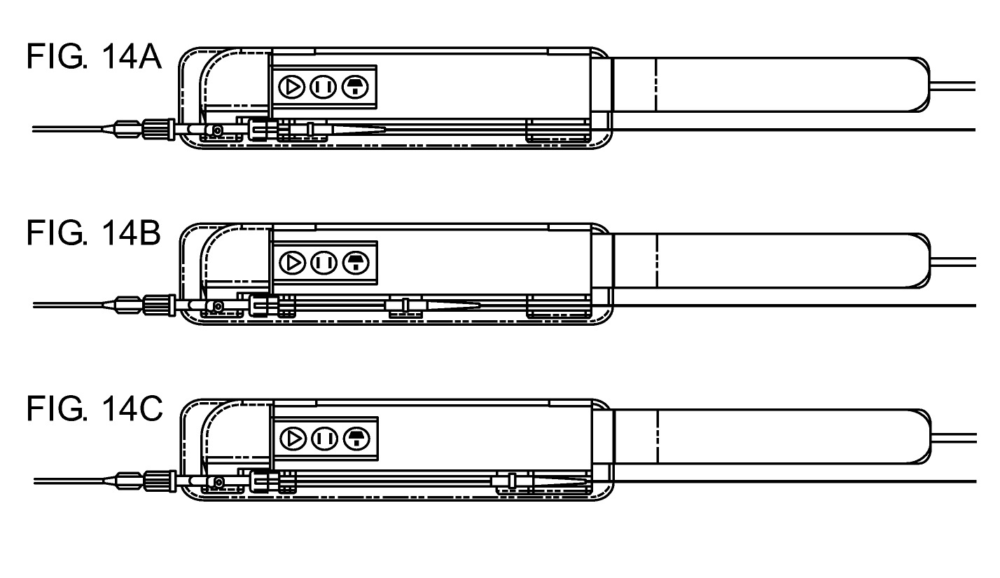

FIG. 13: Sterile PB Device Assembly. Isometric view showing motor drive assembly within sterile sleeve seated in the sterile base assembly with lid open and shut.

FIG. 14: Sterile PB Device Assembly, top view of assembled components, including catheter undergoing pullback. From too to bottom: start position, mid-pullback, completed pullback position.

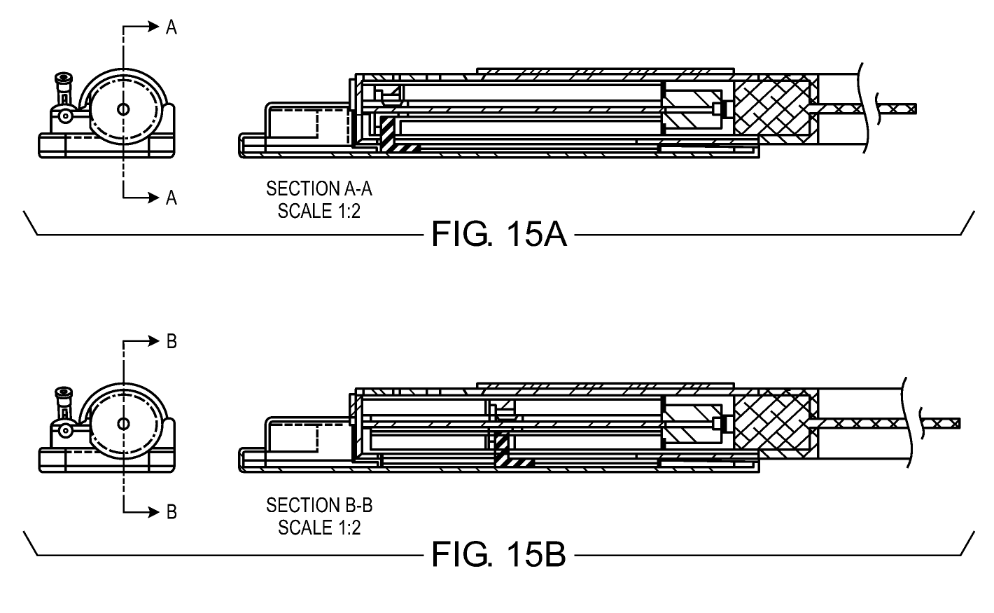

FIG. 15: Sterile PB Device Assembly, Front and section views showing pullback shuttle position within assembly. Section view shows piercing post extending from sterile base unit through sterile sleeve and into reusable motor drive unit.

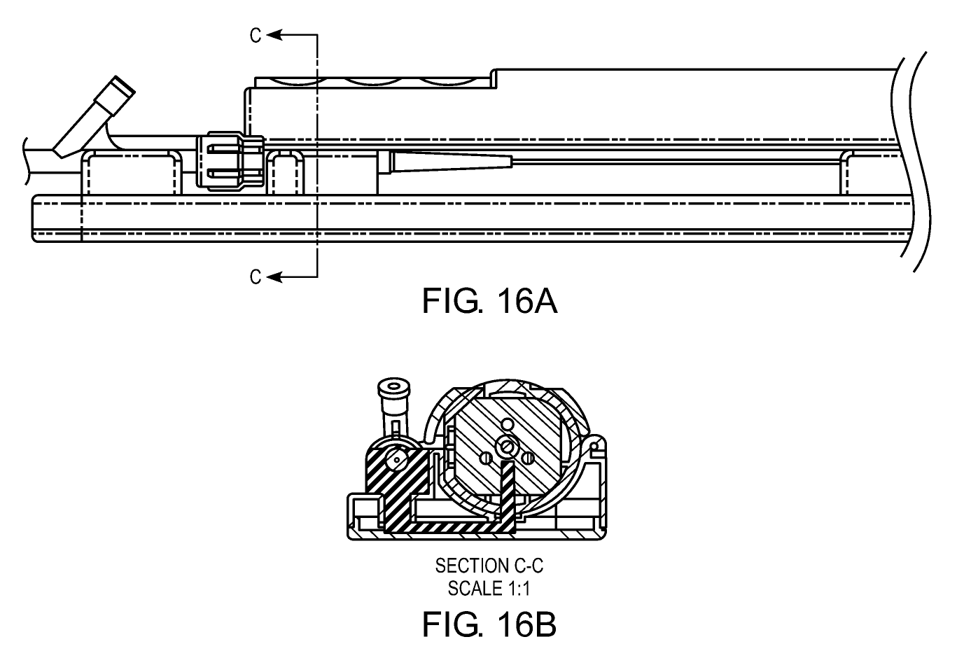

FIG. 16: Sterile PB Device Assembly, Side and section view through pullback shuttle and catheter shuttle. Section view shows piercing post extending from sterile base unit through sterile sleeve and into reusable motor drive unit.

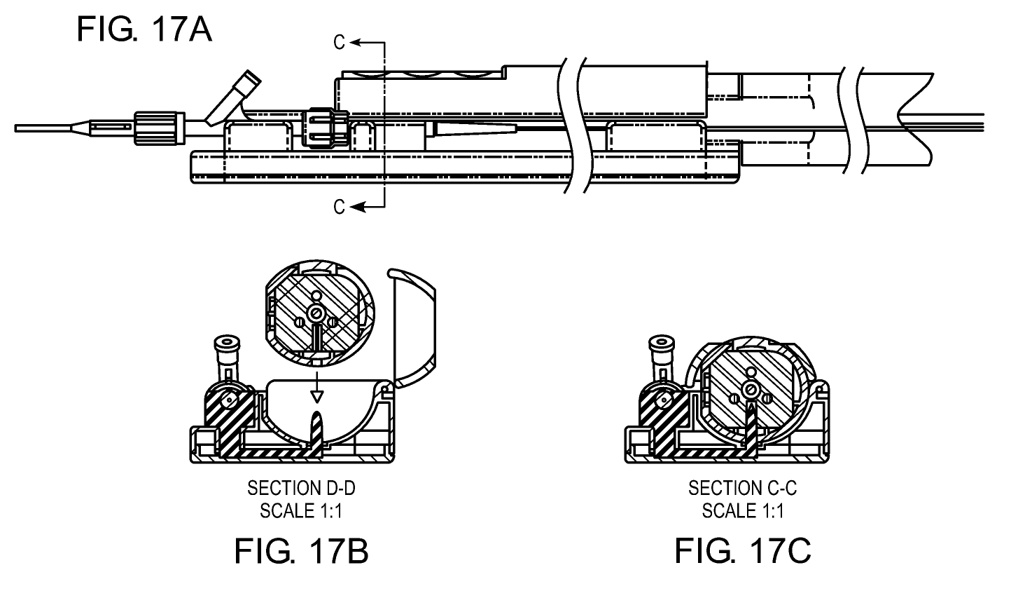

FIG. 17: Similar view to FIG. 16 showing insertion of reusable motor drive assembly within sterile sleeve.

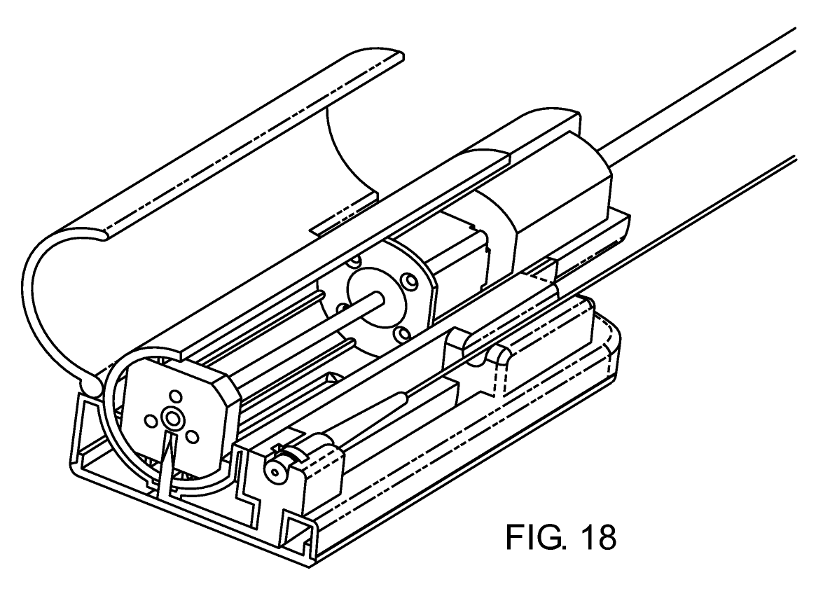

FIG. 18: Cut-away view showing section through pullback & catheter shuttles.

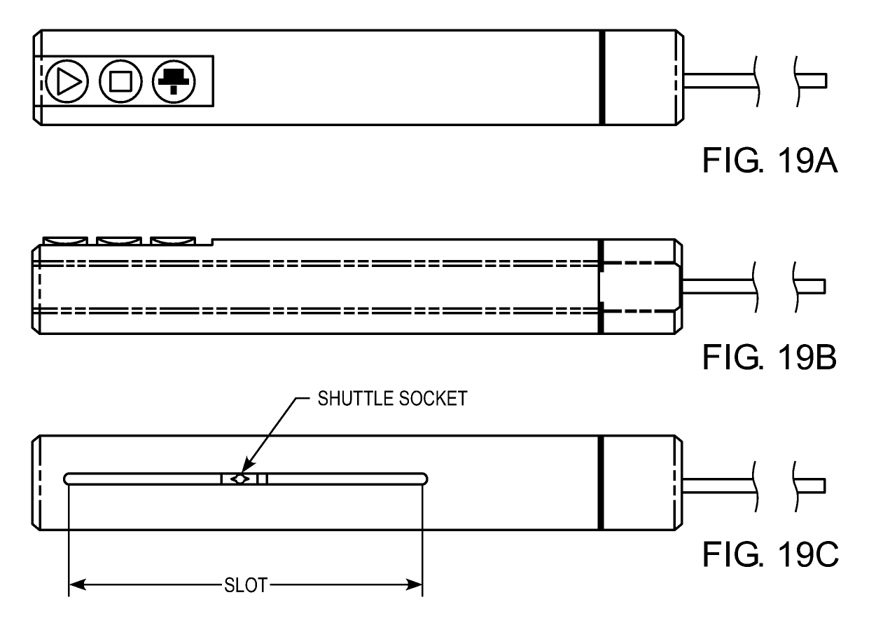

FIG. 19: Top, Side and Bottom views, of motor drive assembly. Bottom view shows slot and shuttle socket.

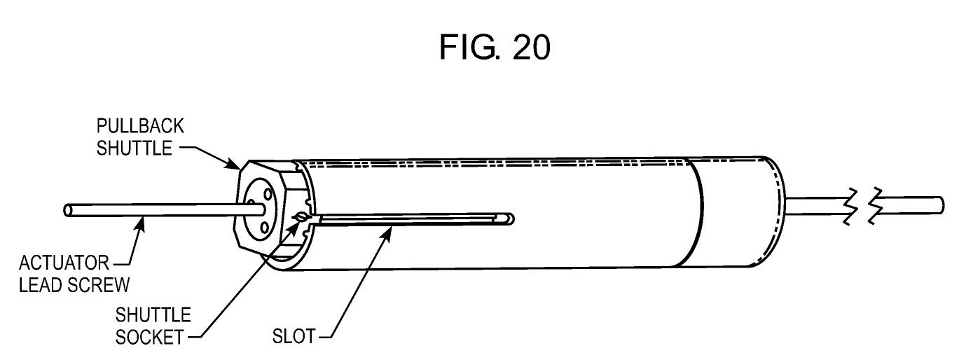

FIG. 20: 3D cut-away view of motor drive assembly showing slot and pullback shuttle coupled to actuator lead screw.

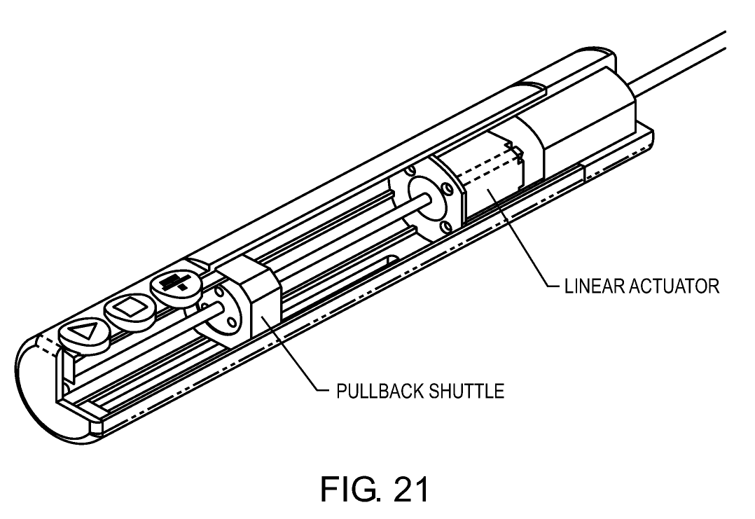

FIG. 21: Isometric cut-away view of motor drive assembly showing slot and pullback shuttle coupled to linear actuator.

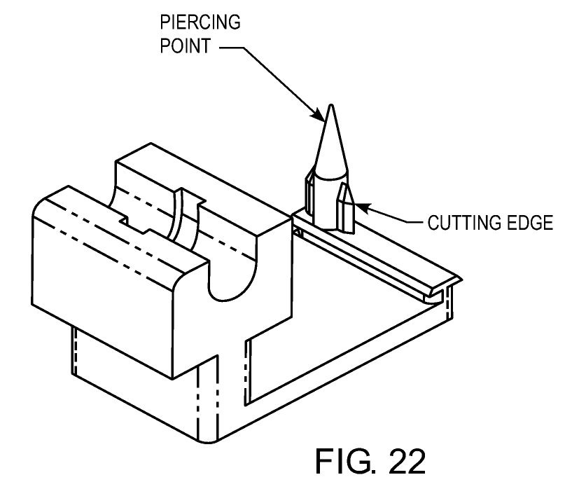

FIG. 22: Isometric view of catheter shuttle component with piercing member.

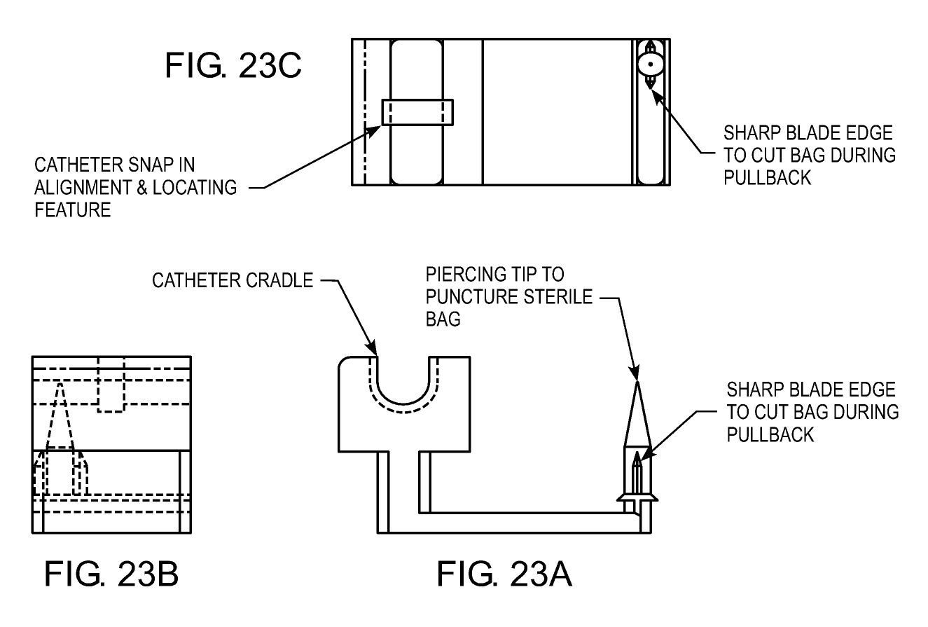

FIG. 23: Front, Side and Top views of catheter shuttle component with piercing member.

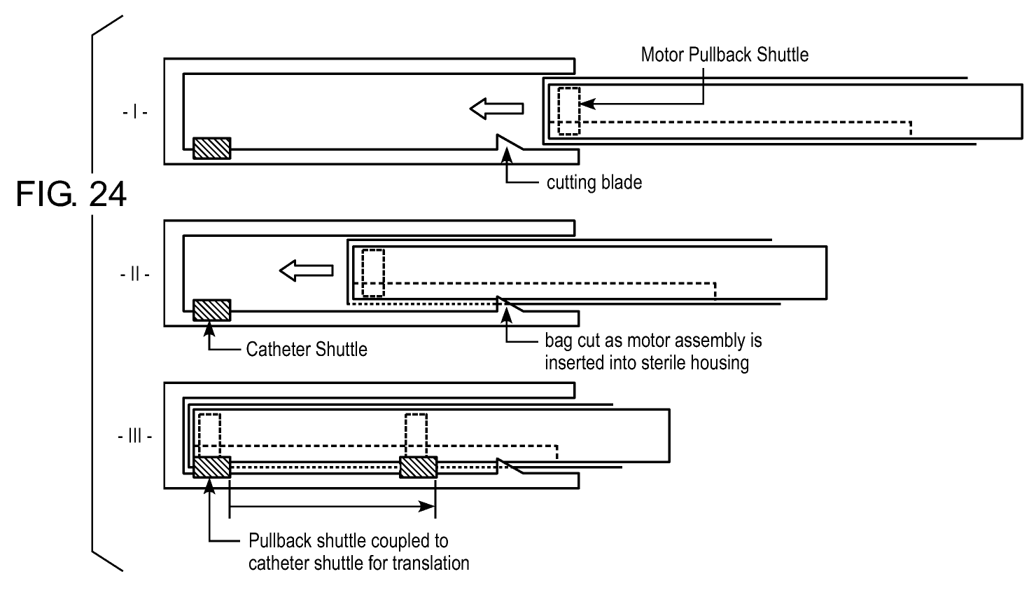

FIG. 24: Embodiment of mechanical coupling mechanism employing a fixed cutting element to split the sterile sleeve during motor drive unit insertion. Coupling of catheter and pullback shuttles may be magnetic or by a physical snap-in feature, where decoupling occurs when the motor unit is pulled out.

### DETAILED DESCRIPTION

The invention overcomes the drawbacks and disadvantages of the prior art by providing catheter pullback device assemblies in which a reusable drive component is enclosed by a sterile barrier enclosure, such as a polymeric sleeve/bag.

Magnetic Coupling-Based Pullback Apparatuses

One embodiment of the invention provides a catheter pullback apparatus, including:

a reusable elongate drive unit comprising a pullback carriage;

a sterile barrier enclosure sized and configured to surround the reusable elongate drive unit;

a base unit sized and configured to receive the reusable elongate drive unit surrounded by the disposable sterile barrier enclosure; and

a catheter shuttle slideably engaged with the base unit and magnetically coupleable to the pullback carriage of the reusable elongate drive unit.

In an assembled state, the apparatus includes:

a reusable elongate drive unit comprising a pullback carriage;

a sterile barrier enclosure conformably surrounding the reusable elongate drive unit;

a base unit sized and configured to receive the reusable elongate drive unit surrounded by the disposable sterile barrier enclosure, in which base unit the same is secured; and

a catheter shuttle slideably engaged with the base unit and magnetically coupled to the pullback carriage of the reusable elongate drive unit.

The sterile barrier enclosure may be for single-use and disposable or may be re-usable following sterilization. The sterile barrier enclosure may, for example, be a polymeric sleeve, preferably closed at one end, or elongated “bag.” The sterile barrier enclosure may be flexible and membranous in form. The sterile barrier enclosure may, for example, be made from a polypropylene or polyethylene.

As used throughout this disclosure, the terms pullback carriage and pullback shuttle are equivalent and refer to the longitudinally displaceable member of the pullback drive unit that couples with the catheter shuttle to provide longitudinal displacement of a catheter.

Various aspects of magnetic coupling-based embodiments of the invention are described below with reference to appended FIGS. 1-9.

FIG. 1 shows an isometric view of a full PB assembly with catheter (and accessories), sterile base assembly, sterile sleeve and reusable motor drive assembly.

FIG. 2 shows an isometric exploded view showing the sterile pullback assembly housing, single-use sterile bag (sleeve) and reusable Motor/Pullback Drive Unit.

FIG. 3 shows an isometric view of the pullback apparatus and engaged catheter in three sequential positions: I. Start; II. Mid-pullback; and III. Full Pullback.

FIG. 4 shows an isometric view of sterile pullback housing assembly with catheter shuttle and track in which the catheter shuttle is slideably engaged.

FIG. 5 shows a sectional isometric view through the pullback assembly housing and catheter shuttle showing the magnetic coupling details.

FIG. 6 shows an isometric view of a pullback shuttle embodiment having rollers and coupling magnets.

FIG. 7 shows an isometric, cut-away view of a reusable magnetic drive motor pullback assembly. The drive unit includes an elongated outer shell (housing), a motor electronics and power supply module, a linear actuator motor, a motor mount plate securing the motor to the shell, a threaded rod operably connected to the motor, a pullback carriage operably connected to the threaded rod by a linear actuator nut, whereby rotation of the rod longitudinally displaces the pullback carriage, pullback carriage guide rails formed in or provided on the inner surface of the shell, and a user control interface operably connected to the motor drive electronic and power supply.

FIG. 8 shows a cross-sectional view through fully assembled pullback unit with catheter showing magnetic coupling between pullback shuttle and catheter shuttle.

FIG. 9 shows an isometric exploded view of a magnetically coupleable reusable pullback motor assembly embodiment.

Catheter shuttle rollers may not be necessary depending upon toughness and friction properties of sterile bag/sleeve material utilized and, thus, are optional. Furthermore, if a suitably strong magnetic field is utilized, the catheter shuttle may not need to be in contact with the sterile sleeve at all. A small separation distance between magnetic elements is desirable for better coupling.

Magnetic coupling is shown in the embodiments of the figures with dual magnets on pullback and catheter shuttle aligned for +/− attractive coupling. Neodymium (Nd—Fe—B alloy) rare-earth magnets are well suited to the present invention, but other permanent magnets may also be used, for example, samarium cobalt, ceramic, AlNiCo, and conductive polymer magnets. In practice, any suitable type, number and/or configuration of magnets may be used.

Alternative embodiments may use magnets on one side only with iron-based magnetic alloys such as ferritic and martensitic 400 series stainless steels. Alternatively, magnetically conductive particles can be mixed in with plastic or insert molded to provide suitable coupling features. Alternatively, electromagnets in the motor housing may be used, which can be switched on during use and off when not in use to limit magnetic interaction with other equipment and devices in the vicinity.

Mechanical Coupling-Based Embodiments

One embodiment of the invention provides a catheter pullback apparatus, including:

a reusable elongate drive unit comprising a pullback carriage;

a sterile barrier enclosure sized and configured to surround the reusable elongate drive unit;

a base unit sized and configured to receive the reusable elongate drive unit surrounded by the disposable sterile barrier enclosure; and

a catheter shuttle comprising a barrier piercing member and slideably engaged with the base unit, said shuttle mechanically coupleable to the pullback carriage of the reusable elongate drive unit by the barrier piercing member.

Thus, the catheter shuttle and pullback carriage are mutually adapted to mechanically couple via the piercing member. In practice, the drive unit surrounded by the sterile barrier is placed in the base unit and concomitantly or thereafter the barrier is pierced by the piercing member of the catheter shuttle to mechanically engage the piercing member with the pullback carriage.

In an assembled state, the apparatus includes:

a reusable elongate drive unit comprising a pullback carriage;

a sterile barrier enclosure conformably surrounding the reusable elongate drive unit;

a base unit sized and configured to receive the reusable elongate drive unit surrounded by the disposable sterile barrier enclosure, in which base unit the same is secured; and

a catheter shuttle comprising a barrier piercing member and slideably engaged with the base unit, said shuttle mechanically coupled to the pullback carriage of the reusable elongate drive unit by the barrier piercing member.

Various aspects of mechanical coupling-based embodiments of the invention are described below with reference to appended FIGS. 10-24.

FIG. 10A shows a top view of the base unit showing piercing tip extending through elongate pullback slot. FIGS. 10B and 10C show isometric views of the base unit with its lid in open and closed positions, respectively. The swinging lid/cover is an optional feature.

FIG. 11 shows an isometric exploded view of the lid, base, catheter shuttle and bottom panel components of the base unit. The catheter shuttle slides back and fort along the pullback slot and guide channels provided by the base and bottom panels.

FIG. 12 shows an isometric view showing a motor drive unit within a sterile sleeve (sterile barrier enclosure) being inserted into the base unit from above. The sharp tip of the piercing member of the catheter shuttle punctures sleeve when the drive unit is seated in the sterile base unit assembly.

FIGS. 13A and 13B shows an isometric view of the drive unit within sterile sleeve seated in the sterile base unit lid open and shut, respectively.

FIGS. 14A-C shows a top view of assembled components, including catheter undergoing pullback. From top to bottom: start position, mid-pullback, completed pullback position.

FIGS. 15A and 15B each show front and sectional views of the pullback assembly with the pullback shuttle at different positions within the apparatus for FIG. 15A versus FIG. 15B. The sectional views show the piercing member (“piercing post”) of the catheter shuttle extending from sterile base unit through the sterile sleeve and into reusable motor drive unit to engage the pullback carriage.

FIGS. 16A and 16B show a side and cross-sectional section view through pullback shuttle (carriage) and catheter shuttle. The cross-sectional view shows the piercing post of the catheter shuttle extending from sterile base unit through sterile sleeve and into reusable motor drive unit to engage the pullback shuttle (carriage).

FIGS. 17A-C show similar views as shown in FIG. 16, further illustrating insertion of the reusable motor drive unit within sterile sleeve into the base unit.

FIG. 18 shows a cut-away, sectional view of the assembled pullback apparatus showing the pullback and catheter shuttles engaged to one another by the piercing member of the catheter shuttle.

FIGS. 19A-C show top, side and bottom views of the drive unit assembly. The bottom of FIG. 19C shows the pullback slot and pullback shuttle socket, which is sized and configured to receive and engage the piercing member of the catheter shuttle of the base unit.

FIG. 20 shows a 3D cut-away view of a drive unit assembly showing the pullback slot and pullback shuttle, including shuttle socket, coupled to the actuator lead screw.

FIG. 21 shows an isometric cut-away view of a drive unit assembly showing the pullback slot therein and pullback shuttle coupled to a linear actuator.

FIG. 22 shows an isometric view of a catheter shuttle component with piercing member and lateral cutting edges to slice the sterile barrier enclosure during longitudinal displacement.

FIGS. 23A-C show a front, side and top view respectively of the catheter shuttle component and its piercing member. The catheter shuttle includes a catheter cradle and/or retainer for engaging the catheter that will be controlled by the pullback apparatus.

FIG. 24 shows an embodiment of a mechanical coupling mechanism employing a fixed cutting element to split the sterile sleeve during motor drive unit insertion. Coupling of catheter and pullback shuttles could be magnetic or via a physical snap-in feature, with decoupling occurring when drive unit is pulled out of the base unit.

The base unit of the apparatuses of the invention may re-usable and sterilizable, for example, by autoclaving. The base unit may for example be metallic and/or polymeric in composition. For example, the housing and body of the catheter shuttle of the base unit may be at least partially made from stainless steel or titanium. The sterile barrier enclosure is preferably a single-use disposable product. The reusable drive unit may be at least partially sterilized between uses using gentle methods such as light surface washing with ethanol, but potential contamination arising from the drive unit is primarily limited by the sterile barrier enclosure.

Any suitable type of linear actuator may be used according to the invention. A simple stepper motor-based linear actuator (such as those provided by Haydon Switch & Instrument, Inc., Waterbury, Conn.) utilizing a lead screw is shown as one example. Alternative drive systems include belt drive, rack & pinion drive, friction wheel/rolling drive, piezo-electric linear actuators, and linear motors and other commonly known linear transducers known in the field of machine design. Both stepper and servo-type motors are suitable as well and may require sensors for shuttle position sensing at start and end of travel locations, or a full linear encoder to provide feedback to drive system.

The pullback drive system may be coupled to a separate power supply or provided from on-board batteries (not shown in figures). External Input/Output communications can also be provided through cabling or wireless means (radio frequency, ultrasonic or infrared based signal transmission).

The invention also provides methods for controlling the position of an endoluminal catheter, that include the steps of:

providing any of the catheter pullback apparatuses described herein;

securing a longitudinally displaceable shaft/element of a catheter to the catheter shuttle; and

selectively longitudinally displacing the pullback carriage to longitudinally displace the catheter shaft/element.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A catheter pullback apparatus, comprising:
a reusable elongate drive unit comprising a pullback carriage;
a sterile barrier enclosure sized and configured to surround the reusable elongate drive unit; and
a base unit sized and configured to receive the reusable elongate drive unit surrounded by the disposable sterile barrier enclosure; and
a catheter shuttle slideably engaged with the base unit and operably coupleable to the pullback carriage of the reusable elongate drive unit.

2. The apparatus of claim 1, wherein the disposable sterile barrier enclosure is polymeric and disposable.

3. The apparatus of claim 1, wherein the catheter shuttle is magnetically coupleable to the pullback carriage of the reusable elongate drive unit.

4. The apparatus of claim 1, wherein the catheter shuttle comprises a piercing member and the catheter shuttle pullback carriage are mutually adapted to mechanically couple via the piercing member.

5. The apparatus of claim 1, wherein the base unit comprises a fixed cutting member sized and configured to slit the sterile barrier enclosure upon insertion of the motor drive unit into the base unit.

6. A method for controlling the position of an endoluminal catheter, comprising the steps of:
providing a catheter pullback apparatus according to claim 1;
securing the a longitudinally displaceable shaft/element of an endoluminal catheter to the catheter shuttle; and
selectively longitudinally displacing the pullback carriage to longitudinally displace the catheter shaft/element.
